(19) United States
(12) Patent Application Publication
(10) Pub. No.: US 2004/0248227 A1
(43) Pub. Date: Dec. 9, 2004

(54) Enzymatic determination of inorganic pyrophosphate

(76) Inventors: Kristian Jansson, Jyvaskyla (FI); Vuokko Jansson, Jyvaskyla (FI)

Correspondence Address: Commissioner for Patents, PO Box 1450, Alexandria, VA, 22313-1450, US

(21) Appl. No.: 10/479,399

(22) PCT Filed: Jun. 11, 2001
(86) PCT No.: PCT/FI01/00549

(51) Int. Cl. C12Q 1/48

(52) U.S. Cl. 435/15

(57) ABSTRACT

A method for determining inorganic pyrophosphate in a sample, which method comprises contacting the sample with an aqueous reagent comprising xanthosine 5′-monophosphate (XMP) or preferrably inosine 5′-monophosphate (IM), xanthosine phosphoribosyltransferase or preferrably hypoxanthine phosphoribosyltransferase, xanthine oxidase, a divalent cation which is preferrably Mg²⁺, and a buffering agent which is preferably tris(hydroxymethyl)aminomethane (Tris); and determining production of hydrogen peroxide as a measure or inorganic pyrophosphate in the sample. Preferrably, the reagent further comprises uricase.

This invention relates to the enzymatic determination of inorganic pyrophosphate in a variety of samples, such as those from nucleic acid primer extension reactions.

In a primer extension reaction, DNA polymerase catalyzes DNA-template-directed extension of the 3′-end of a DNA strand by one nucleotide at a time. The polymerase cannot initiate a chain de novo but requires a DNA or RNA primer able to hybridize to the template strand. During a primer extension reaction, one inorganic pyrophosphate is released for each nucleotide incorporated according to the following equation:

Deoxynucleoside triphosphate+DNAₙ=inorganic pyrophosphate+DNA₍ₙ₊₁₎.

Thus, nucleotide incorporation and primer extension can be determined by determining inorganic pyrophosphate release. On the other hand, the reverse reaction, where the DNA chain is reduced in length, is detrimental to a primer extension reaction. This pyrophosphorolysis reaction depends on the level of inorganic pyrophosphate. Therefore, for optimizing a primer extension reaction, such as a DNA sequencing reaction or a polymerase chain reaction (PCR), the level of inorganic pyrophosphate can be determined.

In an enzymatic bioluminometric inorganic pyrophosphate assay described by Nyrén and Lundin (1985) Analytical Biochemistry, 151, 504-509, ATP formed in an ATP sulfurylase reaction is determined with the light-producing reaction of firefly luciferase. This assay has been frequently used to determine nucleotide incorporation in primer extension reactions, such as those described in WO 89/09283, WO92/16654, WO 93/23562 and US 6210891. However, dATP interferes with the light-producing reaction of firefly luciferase, which severely limits the utility of the assay.

In an assay described by De Groot et al. (1985) Biochemical Journal, 229, 255-260, inorganic pyrophosphate is hydrolyzed by pyrophosphatase to inorganic phosphate, which is reacted with inosine in the presence of purine nucleoside phosphorylase to produce hypoxanthine, which forms uric acid in the presence of xanthine oxidase, and may be measured either by the absorbance of the uric acid, or by the formazan formed when a tetrazolium salt is used as the oxidant.

In JP 3080099, inorganic phosphate is determined by reacting it with inosine in the presence of purine nucleoside phosphorylase, xanthine oxidase and uricase to produce hydrogen peroxide, which is reacted with luminol in the presence of the heme portion of cytochrome c (microperoxidase) to produce chemiluminescence.

In EP 0629705, inorganic phosphate is determined by reacting it with xanthosine in the presence of purine nucleoside phosphorylase and xanthine oxidase to produce hydrogen peroxide, which is reacted with a chromogenic peroxidase substrate in the presence of peroxidase.

In an assay by Salerno et al. (1981) Experientia, 37, 223-224, inosine 5′-monophosphate is determined by reacting it with inorganic pyrophosphate in the presence of hypoxanthine phosphoribosyltransferase and xanthine oxidase to form uric acid measured by its absorbance.

An object of the present invention is to provide a simple enzymatic assay, which is specific for inorganic pyrophosphate, and sensitive enough to be used on small volumes of samples, typical of primer-extension reactions, such as PCR.

Accordingly, this invention provides an assay for inorganic pyrophosphate which comprises: a) reacting inorganic pyrophosphate present in a sample with inosine 5′-monophosphate or xanthosine 5′-monophosphate in the presence of hypoxanthine phosphoribosyltransferase or xanthine phosphoribosyltransferase, xanthine oxidase, Mg²⁺ ion or another divalent cation, a buffering agent, and optionally uricase; and b) determining production of hydrogen peroxide as a measure of inorganic pyrophosphate in the sample.

Determining production of hydrogen peroxide may comprise reacting the hydrogen peroxide with a chemiluminescent peroxidase substrate, such as luminol, in the presence of peroxidase, and measuring chemiluminescence as a measure of production of hydrogen peroxide, and thereby as a measure of inorganic pyrophosphate in the sample.

There are, however, alternative ways for determining production of hydrogen peroxide. For example, by reacting it with a fluorogenic peroxidase substrate, such as 10-acetyl-3,7-dihydroxyphenoxazine (N-acetyldihydroresorufin), in the presence of peroxidase, and measuring fluorescence as a measure of production of hydrogen peroxide, and thereby as a measure of inorganic pyrophosphate in the sample.

To more fully understand this invention, the following enzymatic reactions are shown. Hypoxanthine phosphoribosyltransferase (EC 2.4.2.8) preferentially catalyzes reaction (1). Xanthine phosphoribosyltransferase (EC 2.4.2.22) preferentially catalyzes reaction (2). In reactions (1) and (2), Mg²⁺ ion or another divalent cation is required. Hypoxanthine phosphoribosyltransferase (EC 2.4.2.8) and reaction (1) are preferred. Xanthine oxidase (EC 1.1.3.22) catalyzes reactions (3) and (4). Uricase (EC 1.7.3.3) catalyzes reaction (5). Peroxidase (EC 1.11.1.7) catalyzes reaction (6).

(1) Inosine 5′-monophosphate+inorganic pyrophosphate=phosphoribosyl pyrophosphate+hypoxanthine

(2) Xanthosine 5′-monophosphate+inorganic pyrophosphate pbosphoribosyl pyrophosphate+xanthine

(3) Hypoxanthine+H₂O+O₂=xanthine+H₂O₂

(4) Xanthine+H₂O+O₂=urate+H₂O₂

(5) Urate+2H₂O+O₂=allantoin+CO₂+H₂O₂

(6) Luminol+H₂O₂=3-aminophthalate+N₂+light

A person skilled in the art could optimize enzymatic reactions, such as those above, to adapt a particular mode for carrying out the invention.

The following examples merely illustrate the invention and set forth the best mode contemplated by the inventors for carrying out the invention, but are not to be construed as limiting.

### EXAMPLE 1

An aqueous reagent solution in a volume of 1 ml consisted of:

1000 μM IMP (inosine 5′-monophosphate), sodium salt (Sigma 14500, Lot 14H7813);

150 units of hypoxanthine phosphoribosyl transferase (EC 2.4.2.8), from baker's yeast (Sigma H3389, Lot 103H8040);

0.2 units of xanthine oxidase (EC 1.1.3.22), from buttermilk (Fluka 95493, Lot 405528/1);

0.2 units of uricase (EC 1.7.3.3), from Arthrobacter globiformis (Sigma U7128, Lot 45H1499);

5.2 units of peroxidase (EC 1.11.1.7), from horseradish (Sigma P8375, Lot 10K7430);

50 μM luminol (5-amino-2,3-dihydro-1,4-phthalazinedione), sodium salt (Sigma A4685, Lot 91H38561;

10 mM MgCl₂ (Sigma M1028, Lot 99H89252); and

200 mM Tris-HCl buffer at pH 8.3 (Sigma T5128, Lot 50K5401).

The reagent solution was prepared in water (Aqua sterilisata; Orion Pharma), fresh for each experiment.

### EXAMPLE 2

A sample containing PPᵢ (sodium pyrophosphate, decahydrate; Sigma S6422, Lot 20K0232) in 50 μl of water (Aqua sterilisata; Orion Pharma) was mixed with 50 μl of the reagent solution of Example 1 with or without uricase in a polystyrene test tube (Sarstedt 55476). After 50 seconds of incubation at room temperature, the tube was read in a Berthold 9509 luminometer for 10 seconds, during which time the signal was stable. RLU=relative light units. Data are shown for duplicate reactions (I and II) in Table 1.

TABLE 1

| PPᵢ (pmol) | Uricase | RLU I | RLU II | Average RLU |
|---|---|---|---|---|
| 0 | − | 200 | 197 | 199 |
| 250 | − | 1957 | 2030 | 1993 |
| 0 | + | 221 | 242 | 231 |
| 250 | + | 2957 | 2944 | 2951 |

RLU = relative light units.

Table 1 shows that uricase when included in the reagent solution clearly increased PPᵢ-caused light output.

### EXAMPLE 3

A sample containing various amounts of PPᵢ (sodium pyrophosphate, decahydrate; Sigma S6422, Lot 20K0232) in 50 μl of water (Aqua sterilisata; Orion Pharma) was mixed with 50 μl of the reagent solution of Example 1 in a polystyrene test tube (Sarstedt 55476). After 50 seconds of incubation at room temperature, the tube was read in a Berthold 9509 luminometer for 10 seconds, during which time the signal was stable. RLU=relative light units. Two separate experiments (Expt. 1 and 2) were performed six weeks apart. Data are shown for duplicate reactions (I and U) in Table 2.

TABLE 2

| | | | |
|---|---|---|---|
| PPᵢ (pmol) | RLU I | RLU II | Average RLU |

Expt. 1

| | | | |
|---|---|---|---|
| 0 | 221 | 209 | 215 |
| 5 | 246 | 294 | 270 |
| 25 | 417 | 474 | 445 |
| 50 | 765 | 687 | 726 |
| 250 | 2702 | 2527 | 2615 |
| 500 | 4452 | 3633 | 4043 |

Expt. 2

| | | | |
|---|---|---|---|
| 0 | 190 | 208 | 199 |
| 5 | 223 | 246 | 235 |
| 25 | 406 | 413 | 409 |
| 50 | 702 | 761 | 731 |
| 250 | 2774 | 2680 | 2727 |
| 500 | 4369 | 4172 | 4270 |

RLU = relative light units.

Table 2 shows a linear increase in light output at amounts of PPᵢ from 5 to 250 pmol. The results were reproducible in two separate experiments (Expt. 1 and 2).

### EXAMPLE 4

A complete aqueous PCR reaction mixture in a volume of 100 μl consisted of:

200 μM each of four dNTPs (dkT?, dGTP, dTTP and dCTP) (Roche 1581295);

2.5 units of Taq DNA polymerase (Roche 1647679):

100 pmol of primer L1 (5′-GGTTATCGAAATCAGCCACAGCGCC-3′) and 100 pmol of primer L2 (5′-GATGAGTTCGTGTCCGTACAACTGG-3′) flanking a 500-base pair sequence of lambda phage DNA;

50 pg (1×10⁶ copies) of lambda phage DNA (Sigma D 9768); and

10 mM Tris-HCl buffer, 1.5 mM MgCl₂, 50 mM KC, pH 8.3 (Roche 1647679).

Various combinations of the PCR reaction mixture components prepared in water (Aqua sterilisata; Orion Pharma), in a volume of 100 μl, including the complete reaction mixture, were placed in a Bio-Rad Gene Cycler thermal cycler and subjected to 25 cycles of 94° C. for 30 seconds, 60° C. for 30 seconds and 72° C. for 30 seconds. After thermal cycling, a sample consisting of 5 μl of the complete PCR reaction mixture or a combination of components thereof and 45 μl of water (Aqua sterilisata; Orion Pharma) was mixed with 50 μl of the reagent solution of Example 1 in a polystyrene test tube (Sarstedt 55476). After 50 seconds of incubation at room temperature, the tube was read in a Berthold 9509 luminometer for 10 seconds, during which time the signal was stable. RLU=relative light units. The reactions were assembled in duplicate (I and II) as shown in Table 3.

TABLE 3

| PCR mixture components | RLU I | RLU II | Average RLU |
|---|---|---|---|
| Buffer | 208 | 242 | 225 |
| Buffer + Taq | 213 | 203 | 208 |
| Buffer + dNTPs | 315 | 287 | 301 |
| Buffer + dNTPs + Taq | 265 | 272 | 269 |
| Buffer + dNTPs + primers | 267 | 295 | 281 |
| Buffer + dNTPs + primers + Taq | 754 | 721 | 737 |
| Buffer + dNTPs + primers + DNA | 318 | 281 | 299 |
| Buffer + dNTPs + primers + | 2261 | 2369 | 2315 |
| DNA + Taq | | | |
| Buffer + dNTPs + primers + DNA + | 202 | 200 | 201 |
| Taq + PPᵢase^{b} | | | |
| RLU = relative light units. | | | |
| ^{b}After thermal cycling, 1 unit of inorganic pyrophosphatase (PPᵢase) from baker's yeast (Sigma I1643) was added in 5 μl of water (Aqua sterilisata; Orion Pharma) to a reaction tube containing 100 μl of the complete PCR reaction mixture. After 10 minutes of incubation at room temperature, a sample consisting of 5 μl of the reaction mixture and 45 μl of water (Aqua sterilisata; Orion Pharma) | | | |
| # was mixed with 50 μl of the reagent solution of Example 1 and processed as above. | | | |

Table 3 shows that the release of PPᵢ due to the amplification of lambda DNA was clearly detectable in as little as 5 μl of the complete PCR reaction mixture. Taq-polymerase-catalyzed primer dimer formation during thermal cycling was another significant source of PPᵢ. Addition of PPᵢase to the PCR mixture after thermal cycling reduced the signal to the background level. Therefore, observed increases in light output were caused solely by PPᵢ.

1. An assay for inorganic pyrophosphate, characterized in that it comprises:
(a) reacting inorganic pyrophosphate present in a sample with inosine 5′-monophosphate or xanthosine 5′-monophosphate in the presence of hypoxanthine phosphoribosyltransferase or xanthine phosphoribosyltransferase, xanthine oxidase, Mg⁺ ion or another divalent cation, a buffering agent, and optionally uricase; and
(b) determining production of hydrogen peroxide as a measure of inorganic pyrophosphate in the sample.
